(19) United States
(12) Patent Application Publication
(10) Pub. No.: US 2025/0032037 A1
(43) Pub. Date: Jan. 30, 2025

(54) METHOD FOR ENRICHING EPILEPTIFORM DISCHARGES AND PREDICTING SOZ DURING EPILEPSY INTERICTAL PERIOD

(71) Applicant: SOUTH CHINA NORMAL UNIVERSITY, Guangzhou (CN)

(72) Inventors: Jiuxing LIANG, Guangzhou (CN); Guozhao RUAN, Guangzhou (CN); Lizhang ZENG, Guangzhou (CN); Yongxin YANG, Guangzhou (CN); Lidao XU, Guangzhou (CN); Xuchu WENG, Guangzhou (CN)

(21) Appl. No.: 18/781,629

(22) Filed: Jul. 23, 2024

(30) Foreign Application Priority Data
Jul. 26, 2023 (CN) 202310923223.4

(51) Int. Cl. A61B 5/372; A61B 5/00; A61B 5/293; G06N 3/045

(52) U.S. Cl. CPC A61B 5/372; A61B 5/293; A61B 5/7221; A61B 5/7246; A61B 5/725; A61B 5/7267; A61B 5/7275; G06N 3/045

(57) ABSTRACT

The disclosure provides a method for enriching epileptiform discharges and predicting SOZ during epilepsy interictal period, including the following steps: obtaining stereotactic electroencephalogaphy signals of patient during the epilepsy interictal period; preprocessing the stereotactic electroencephalogaphy signals to obtain processed stereotactic electroencephalogaphy signals; dividing the processed stereotactic electroencephalogaphy signals into a training set and a test set, dividing the training set into a plurality of signal segments by using a sliding window, and performing a self-supervised reconstruction training on the signal segments based on a Transformer encoder model to obtain a trained Transformer encoder model; inputting the test set into the trained Transformer encoder model, obtaining reconstructed values of each of the signal segments, comparing the reconstructed values of each of the signal segments with values of the stereotactic electroencephalogaphy signals to obtain deviation values of each of the signal segments from the background signals.

### CROSS-REFERENCE TO RELATED APPLICATIONS

This application claims priority to Chinese Patent Application No. 202310923223.4, filed on Jul. 26, 2023, the contents of which are hereby incorporated by reference.

### TECHNICAL FIELD

The disclosure belongs to the technical field of medical electrophysiological auxiliary evaluation and examination, and in particular relates to a method for enriching epileptiform discharges and predicting SOZ during epilepsy interictal period.

### BACKGROUND

The evaluation of epileptogenic zone is the key to the success of epilepsy surgery. Unfortunately, there is no method to directly measure epileptogenic zone at present, and the seizure onset zone (SOZ) is usually used as an indirect measurement of epileptogenic zone. Stereotactic electroencephalography (SEEG) has always been an important diagnostic tool for clinicians to locate SOZ. At present, the main biomarkers used in clinic are: Spikes and high frequency oscillations (HFOs). The identification of these biomarkers is usually done by clinicians' manual vision, but this method is too time-consuming and subjective. There are many traditional automatic detection methods, but they all encounter a series of controversial problems such as feature selection, feature combination and feature threshold range selection. The method of deep learning is capable of avoiding the trouble of artificial feature extraction, but it is very difficult to directly classify SOZ between SEEG interictal period. Because the data signal of SEEG interictal period is very long, when the data is directly input into some common time series models, there will be serious “forgetting”, and the final prediction results are biased towards random values. Moreover, there are few “effective” signals during the interictal period, and the proportion of Spikes and HFOs in the signals is very low. Most of the signals are redundant background signals. SEEG interictal period has a long data signal and a low effective signal ratio, so the SOZ classification condition of SEEG directly using deep learning model is very poor. Therefore, it is urgent to develop a method for enriching epileptiform discharges and predicting SOZ during epilepsy interictal period.

### SUMMARY

In order to solve the above technical problems, the disclosure provides a method for enriching epileptiform discharges and predicting SOZ during epilepsy interictal period, which improves the classification accuracy of SOZ and may assist doctors in judging the epileptogenic zone.

In order to achieve the above objectives, the present disclosure provides a method for enriching epileptiform discharges and predicting SOZ during epilepsy interictal period, including the following steps:
obtaining stereotactic electroencephalogaphy signals of patient during the epilepsy interictal period;
preprocessing the stereotactic electroencephalogaphy signals to obtain processed stereotactic electroencephalogaphy signals;
dividing the processed stereotactic electroencephalogaphy signals into a training set and a test set, dividing the training set into a plurality of signal segments by using a sliding window, and performing a self-supervised reconstruction training on the signal segments based on a Transformer encoder model to obtain a trained Transformer encoder model;
inputting the test set into the trained Transformer encoder model, obtaining reconstructed values of each of the signal segments, comparing the reconstructed values of each of the signal segments with values of the stereotactic electroencephalogaphy signals to obtain deviation values of each of the signal segments from the background signals;
setting threshold values of the deviation values, extracting all signal segments exceeding the threshold values of the deviation values, and performing processing based on the signal segments to obtain averaged signal segments; and
inputting the averaged signal segments into a bidirectional long short term memory recursive neural network model to classify and evaluate the stereotactic electroencephalogaphy signals, and completing epileptiform discharge enrichment and SOZ prediction during the epilepsy interictal period.

Optionally, a method for obtaining the stereotactic electroencephalogaphy signals during the epilepsy interictal period of the patient includes: placing stereotactic electroencephalogaphy electrodes in the patient by adopting stereotactic technology, setting a sampling rate, and obtaining the stereotactic electroencephalogaphy signals during the epilepsy interictal period of the patient.

Optionally, a method of preprocessing the stereotactic electroencephalogaphy signals to obtain the processed stereotactic electroencephalogaphy signals includes:
based on the stereotactic electroencephalogaphy signals, adopting a bipolar reference to minimize correlation between two adjacent channels, then performing high-pass filtering on the stereotactic electroencephalogaphy signals, and performing an unified resampling, and finally the stereotactic electroencephalogaphy signals are subjected to Z-score standardization to obtain the processed stereotactic electroencephalogaphy signals.

Optionally, before adopting the sliding window, masking and position coding need to be performed on the sliding window, specifically including: masking a middle position of the sliding window with 0; and using sine and cosine functions for position coding.

Optionally, dividing the training set into a plurality of signal segments by adopting the sliding window, and performing the self-supervised reconstruction training on the signal segments based on the Transformer encoder model, and obtaining the trained Transformer encoder model by following methods:

$$\begin{matrix}{PE}_{( pos + k , 2 i )} = sin [ ( pos + k ) / {10000}^{2 i / {d}_{model}} ] \\ {PE}_{( pos + k , 2 i + 1 )} = cos [ ( pos + k ) / {10000}^{2 i / {d}_{model}} ] \\ Attention ( Q , K , V ) = softmax ( \frac{{QK}^{T}}{\sqrt{{d}_{k}}} ) V ,\end{matrix}$$

where PE represents position coding, pos represents position, i represents dimension, d_{model} represents dimension size, sine function is used for even dimensions, and cosine function is used for odd dimensions; Q is a query vector, K is a vector of correlation between queried information and other information, V is a vector of the queried information, and de is dimension size.

Optionally, a method for comparing the reconstructed values of each of the signal segments with the values of the stereotactic electroencephalogaphy signals to obtain the deviation values of each of the signal segments from the stereotactic electroencephalogaphy signals is as follows:

$$MSE = \frac{1}{n} {\sum }_{i = 1}^{n} {( {Y}_{i} - {\hat{Y}}_{i} )}^{2} ,$$

where MSE is a mean square error function, n is a number of signal points in the signal segments, and Yᵢ is an i-th real signal and Ýᵢ is an i-th predicted signal.

Optionally, a method for performing the processing based on the signal segments to obtain the averaged signal segments includes: converting the signal segments by using a smooth nonlinear energy algorithm to obtain converted signal segments; performing average processing on the converted signal segments to obtain the averaged signal segments.

Optionally, the averaged signal segments are input into the bidirectional long short term memory recursive neural network model to classify the stereotactic electroencephalogaphy signals, where the bidirectional long short term memory recursive neural network model introduces a bidirectional propagation mechanism and an attention mechanism on a basis of a long short term memory network, specifically including:

$$\begin{matrix}{f}_{t} = σ ( {W}_{f} \cdot [ {x}_{t} , {h}_{t - 1} ] + {b}_{f} ) \\ {i}_{t} = σ ( {W}_{i} \cdot [ {x}_{t} , {h}_{t - 1} ] + {b}_{i} ) \\ {g}_{t} = tanh ( {W}_{c} \cdot [ {x}_{t} , {h}_{t - 1} ] + {b}_{c} ) \\ {c}_{t} = {i}_{t} {g}_{t} + {f}_{t} {c}_{t - 1} \\ {o}_{t} = σ ( {W}_{o} \cdot [ {x}_{t} , {h}_{t - 1} ] + {b}_{o} ) \\ {h}_{t} = {o}_{t} tanh ( {c}_{t} ) \\ {h}_{i} = [ {\vec{h}}_{i} ⊕ {\overleftarrow{h}}_{i} ] ,\end{matrix}$$

where σ is a sigmod function, xₜ is an input at a t-th time, and hₜ₋₁ is a hidden layer vector at a last time; fₜ is a forgetting gate, W_{f} is a learning weight of the forgetting gate, and b_{f} is a learning weight bias of the forgetting gate; iₜ and gₜ are two branch lines of an input gate, cₜ is an output of the input gate, Wᵢ and W_{c} are learning weights of the input gate, and b; and be are learning weight biases of the input gate; oₜ is an output gate, Wₒ is a learning weight of the output gate, and bₒ is a learning weight bias of the output gate; hₜ is a hidden layer vector calculated at a current t time; \vec{h}ᵢ and [symbol] are hidden layer vectors from front to back and from back to front, respectively.

Optionally, a method for evaluating the stereotactic electroencephalogaphy signals includes:

$$\begin{matrix}Accuary = \frac{TP + TN}{TP + FP + FN + TN} \\ Sensitivity = \frac{TP}{TP + FN} \\ Specificity = \frac{TN}{TN + FP}\end{matrix}$$

where TP represents true positive, TN represents true negative, FP represents false positive, FN represents false negative, Accuracy is accuracy, Sensitivity is sensitivity and Specificity is specificity.

The technical effect of the disclosure are as follows: the disclosure provides a method for enriching epileptiform discharges and predicting SOZ during epilepsy interictal period, which solves the problem that the classification result of seizure onset zone by directly using deep learning patterns is poor due to long data signals and low effective signal ratio in the interictal period of stereo-electroencephalography, improves the signal-to-noise ratio of stereo-electroencephalography data, effectively improves the classification accuracy of seizure onset zone, and assists doctors in judging seizure onset zone.

### BRIEF DESCRIPTION OF THE DRAWINGS

The accompanying drawings, which constitute a part of this application, are used to provide a further understanding of this application. The illustrative examples and descriptions of this application are used to explain this application, and do not constitute an improper limitation of this application. In the attached drawings:

FIG. 1 is a schematic flow chart of a method for enriching epileptiform discharges and predicting SOZ during epilepsy interictal period according to an embodiment of the present disclosure;

FIG. 2 is a schematic diagram of masking and position coding of SEEG window data according to an embodiment of the present disclosure;

FIG. 3 is a schematic diagram of the SEEG original values and the deviation values according to the embodiment of the present disclosure;

FIG. 4 is an enriched epileptiform discharge fragment according to an embodiment of the present disclosure;

FIG. 5 is a schematic diagram of the average value of signal segments in an embodiment of the present disclosure; and

FIG. 6 is a schematic diagram of the direct classification result and the result after improving the signal-to-noise ratio according to the embodiment of the present disclosure.

### DETAILED DESCRIPTION OF THE EMBODIMENTS

It should be noted that the embodiments in this application and the features in the embodiments may be combined with each other without conflict. The present application will be described in detail with reference to the attached drawings and embodiments.

It should be noted that the steps shown in the flowchart of the accompanying drawings may be executed in a computer system such as a set of computer-executable instructions, and although the logical order is shown in the flowchart, in some cases, the steps shown or described may be executed in a different order from here.

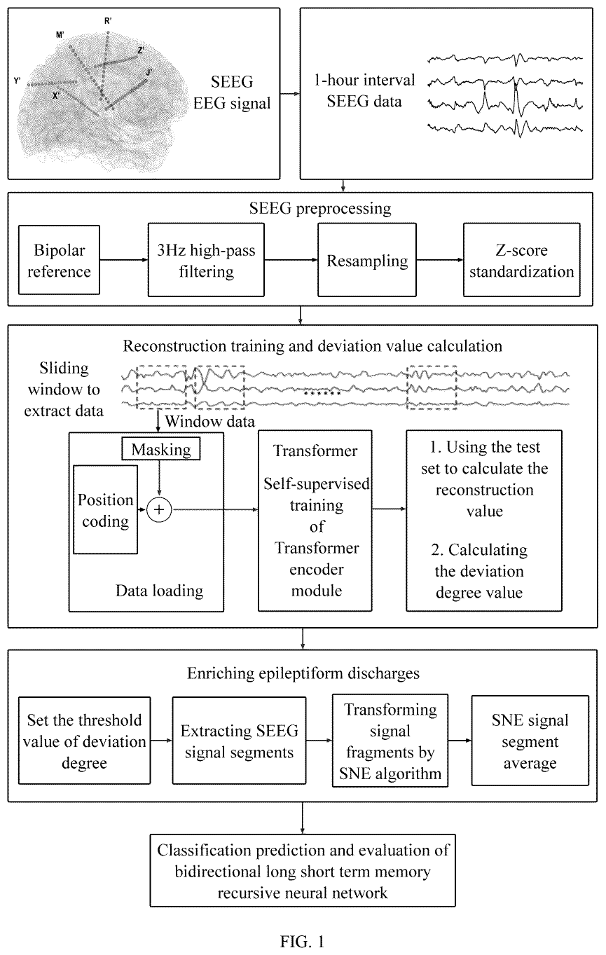

As shown in FIG. 1, this embodiment provides a method for enriching epileptiform discharges and predicting SOZ during epilepsy interictal period, including the following steps:
obtaining stereotactic electroencephalogaphy signals of patient during the epilepsy interictal period;
preprocessing the stereotactic electroencephalogaphy signals to obtain processed stereotactic electroencephalogaphy signals;
dividing the processed stereotactic electroencephalogaphy signals into a training set and a test set, dividing the training set into a plurality of signal segments by using a sliding window, and performing a self-supervised reconstruction training on the signal segments based on a Transformer encoder model to obtain a trained Transformer encoder model;
inputting the test set into the trained Transformer encoder model, obtaining reconstructed values of each of the signal segments, comparing the reconstructed values of each of the signal segments with values of the stereotactic electroencephalogaphy signals to obtain deviation values of each of the signal segments from the background signals;
setting threshold values of the deviation values, extracting all signal segments exceeding the threshold values of the deviation values, and performing processing based on the signal segments to obtain averaged signal segments; and
inputting the averaged signal segments into a bidirectional long short term memory recursive neural network model to classify and evaluate the stereotactic electroencephalogaphy signals, and completing epileptiform discharge enrichment and SOZ prediction during the epilepsy interictal period.

SEEG electrodes are implanted in the patient with intractable epilepsy by stereotactic technique. Each electrode has 8-18 electrode points, and the sampling rate is 1000 or 2000 Hertz (Hz). The relatively stable SEEG signals of 3000-5000 seconds are selected, and the electroencephalogaphy signals during sleep and waking periods may be used.

### Preprocessing Method and Flow of SEEG Data

The SEEG signal adopts bipolar reference to minimize the correlation between two adjacent channels, then high-pass filtering at 1 Hz, and then uniformly resampling to J Hz, for example, high-pass filtering at 4 Hz, and then uniformly resampling to 1000 Hz. Before training, SEEG data need to be standardized, and the Z-score is used to map the data to a normal distribution with a mean of 0 and a standard deviation of 1. The Z-score formula is as follows:

$$z = \frac{x - η}{σ}$$

where x is the input SEEG signal, n represents the mean value of SEEG signal, and σ represents the standard deviation of SEEG signal.

Using self-monitoring model to train SEEG interictal period data, including:
(1) model selection and data loading: A % SEEG signals are intercepted as a training set, and the rest data is used as a test set, and the test set is used to calculate the deviation values. Sliding window is used to extract data, the window length is W and the number of sliding steps is D. For example, 20% SEEG signals are intercepted as the training set, and the remaining data is used as the test set, and the test set is used to calculate the deviation values. Sliding window is used to extract data, the window length is 256, and the number of sliding steps is 1. Then, the disclosure uses the Transformer coder to carry out self-supervised reconstruction training on the SEEG signals, and the Transformer is originally designed for natural language processing, so the model input parameter is the word Embedding. In order to align the SEEG signal data with the Transformer input format, the disclosure defines the sliding window length W of SEEG signal as the sentence length, and the signals collected by a single patient at the same time point is defined as the word Embedding, that is, d_{model}. In this way, SEEG signals are capable of being transformed into data format of natural language processing and trained by Transformer.
(2) Training process, before the SEEG window data is input into the model, it is necessary to mask the window data and position code PE. masking is to mask the middle part of window data with a length of h with 0. The sine and cosine functions are selected for the position coding. The smaller the position index value is, the longer the wavelength is, and the position coding corresponding to each position is unique. The position coding formula is as follows:

$$\begin{matrix}{PE}_{( pos , 2 i )} = sin [ pos / {10000}^{2 i / {d}_{model}} ] \\ {PE}_{( pos , 2 i + 1 )} = cos [ pos / {10000}^{2 i / {d}_{model}} ] ,\end{matrix}$$

where pos is the position, i is the dimension, d_{model} is the dimension size, sine function is used for even dimensions, and cosine function is used for odd dimensions.

The position codes and the masked data are added, and the result of the addition is sent to the N-layered Transformer encoder. The self-attention formula in the Transformer is:

$$Attention ( Q , K , V ) = softmax ( \frac{{QK}^{T}}{\sqrt{{d}_{k}}} ) V ,$$

where Q is the query vector, K is the vector of the correlation between the queried information and other information, V is the vector of the queried information, and dₖ is the dimension size.

The output of the masking part is the masked reconstructed values, which is retained, and the output of other positions is discarded. The reconstructed values are compared with the original data before masking, and the mean square error (MSE) is used to calculate the loss. The formula of MSE is as follows:

$$MSE = \frac{1}{n} {\sum }_{i = 1}^{n} {( {Y}_{i} - {\hat{Y}}_{i} )}^{2} ,$$

where n is the number of signal points in the signal segments, Yᵢ is the i-th real signal and Ŷᵢ is the i-th predicted signal.

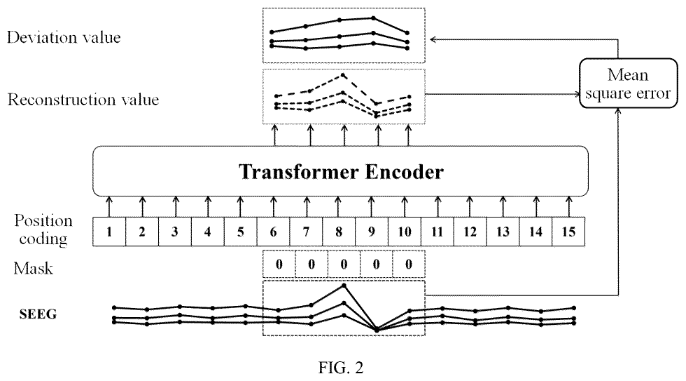

For example, masking is to mask the middle part of the window data with a length of 16 with 0, add the masked data with the position codes, and send the result of the addition to a two-layered Transformer encoder. The design details are shown in FIG. 2.

Calculating the deviation value specifically includes: using the same sliding window as the training set to extract the window data of the test set, performing masking and position coding for the window data, inputting the trained model, and outputting the model as a reconstructed value. The reconstructed values are compared with the original data before masking, and the mean square error is obtained. The larger the mean square error value, the greater the difference between the signal of the sliding window and the background signal, and the smaller the mean square error value, the smaller the difference. Therefore, the disclosure defines the mean square error value as the deviation value between the sliding window and the background signal.

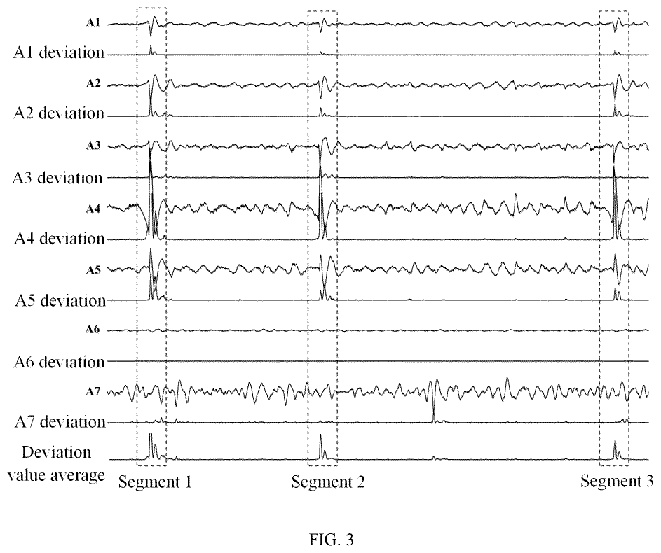

Enriching epileptiform discharges specifically includes: after calculating the deviation values, averaging the deviation values, standardizing the deviation values by using Z score, and marking all peaks of the average abnormal value, and setting the threshold at 3, and the peaks greater than 3 standard deviations will be defined as deviation anomalies. Taking the peak as the midpoint, a signal segment with a length of 200 is intercepted on the SEEG signals, as shown in FIG. 3. Compared with using SEEG signal directly for classification, this method of intercepting signals may greatly improve the signal-to-noise ratio of SEEG data.

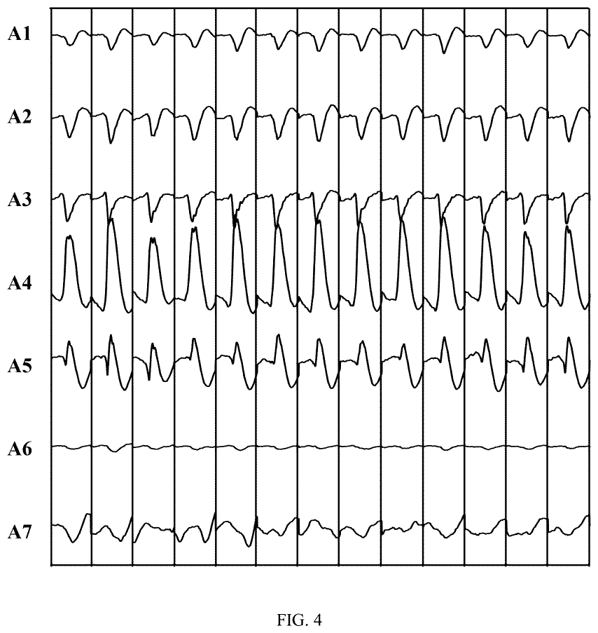

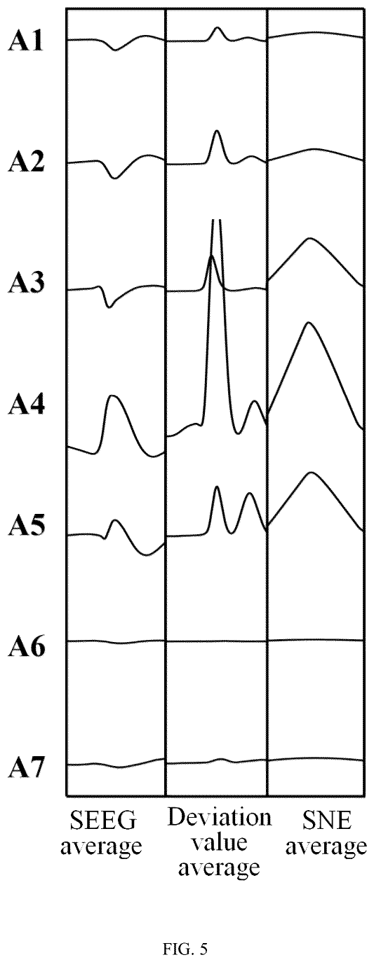

The averaging of the signal segments includes: after the signal segments are intercepted, as shown in FIG. 4, the signal segments are converted by using the smooth nonlinear energy (SNE) algorithm. Directly averaging SEEG signal segments will cause some high-frequency information to be lost, and SNE may effectively retain high-frequency information. SNE is divided into two steps: nonlinear energy operator (NEO) and windowing. NEO is an operator for estimating the energy content of a linear oscillator, which use the frequency and amplitude information of the signal, not just the amplitude information. Meanwhile, the output is proportional to the product of the amplitude and frequency of the input signal, which may highlight the high-frequency components and suppress the low-frequency components. For the discrete signal x(n), the expression of NEO is: Ψ[x(n)]=x² (n)−x(n−1)×(n+1). In order to further improve the ability of NEO to represent non-stationary signals, a window function is usually added to NEO algorithm, and the formula is Y's [x(n)]=ω(n)*Ψ[x(n)], where ω(n) is a triangular window function, * represents convolution operation, and the output Y's [x(n)] is SNE value, and the input and output are equal in length, all of which are M. The input and output lengths of SNE are equal, all of which are 200. After SNE conversion, all SNE signal segments on a single electrode point are averaged, and finally each electrode point obtains a one-dimensional SNE signal segment with a length of 200. As shown in FIG. 5.

The disclosure selects a bidirectional long short term memory recursive neural network to classify one-dimensional SNE signal segments, and the bidirectional long short term memory recursive neural network introduces a bidirectional propagation mechanism on the basis of a long short term memory network (LSTM). The formula of LSTM module is as follows:

$$\begin{matrix}{f}_{t} = σ ( {W}_{f} \cdot [ {x}_{t} , {h}_{t - 1} ] + {b}_{f} ) \\ {i}_{t} = σ ( {W}_{i} \cdot [ {x}_{t} , {h}_{t - 1} ] + {b}_{i} ) \\ {g}_{t} = tanh ( {W}_{c} \cdot [ {x}_{t} , {h}_{t - 1} ] + {b}_{c} ) \\ {c}_{t} = {i}_{t} {g}_{t} + {f}_{t} {c}_{t - 1} \\ {o}_{t} = σ ( {W}_{o} \cdot [ {x}_{t} , {h}_{t - 1} ] + {b}_{o} ) \\ {h}_{t} = {o}_{t} tanh ( {c}_{t} )\end{matrix}$$

where σ is a sigmod function, xₜ is an input at a t-th time, and hₜ₋₁ is a hidden layer vector at a last time; fₜ is a forgetting gate, W_{f} is a learning weight of the forgetting gate, and b_{f} is a learning weight bias of a forgetting gate; iₜ and gₜ are two branch lines of an input gate, cₜ is an output of the input gate, Wᵢ and W_{c} are learning weights of the input gate, and bᵢ and b_{c} are learning weight biases of the input gate; oₜ is an output gate, Wₒ is a learning weight of the output gate, and bₒ is a learning weight bias of the output gate.

By adding the back-and-forth bidirectional propagation mechanism, the back-and-forth bidirectional information of the time series signal may be used more effectively, and the formula is as follows:

$${h}_{i} = [ {\vec{h}}_{i} ⊕ {\overleftarrow{h}}_{i} ] ,$$

where \vec{h}ᵢ and [symbol] represent hidden layer vectors from front to back and from back to front respectively.

The evaluation details of model performance are as follows.

True negative (TN) refers to the situation that it is actually negative and the model predicts negative. In the present disclosure, it means that it is actually non-SOZ but the model is predicts non-SOZ.

False positive (FP) refers to the situation that it is actually negative and the model predicts positive. In the present disclosure, it means that the situation that it is actually non-SOZ and the model predicts SOZ.

False negative (FN) refers to the situation that it is actually a positive and the model predicts negative. In the present disclosure, it means that the situation that it is actually SOZ but the model predicts non-SOZ.

True positive (TP) refers to the situation that it is actually positive and the model predicts positive. In the present disclosure, it means that it is actually SOZ and the model also predicts SOZ.

Accuracy refers to the probability that all the samples predicted to be positive are actually positive; Sensitivity refers to the proportion of all cases diagnosed as positive, also known as true positive rate (TPR); Specificity refers to the correct proportion of all negative samples, which measures the classifier's ability to identify negative samples. The formulas are as follows:

$$\begin{matrix}Accuary = \frac{TP + TN}{TP + FP + FN + TN} \\ Sensitivity = \frac{TP}{TP + FN} \\ Specificity = \frac{TN}{TN + FP}\end{matrix}$$

where TP is true positive, TN is true negative, FP is false positive, FN is false negative, Accuracy is accuracy, Sensitivity is sensitivity and Specificity is specificity.

In the disclosure, the accuracy, sensitivity and specificity are used as evaluation indicators of the machine learning classification algorithm.

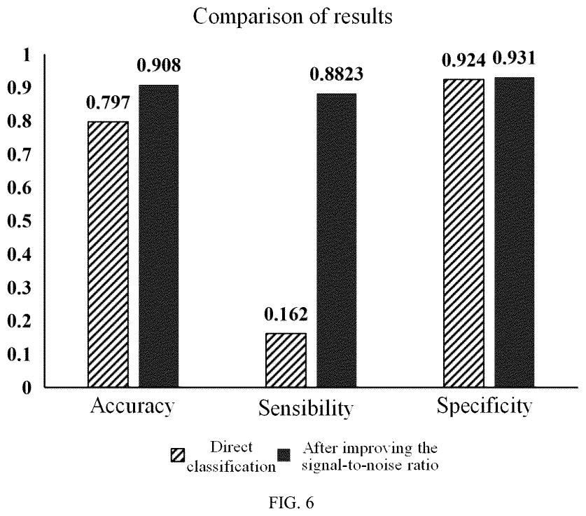

The patients are randomly divided into five groups, and 50% cross-validation is done among patients. Four groups of patients are used as training and the other group as testing. Then the test set and training set are input into the bidirectional long short term memory recursive neural network for training, testing and evaluation. Finally, the results of direct classification are compared with those after improving the signal-to-noise ratio of SEEG. As shown in FIG. 6.

The above is only the preferred embodiment of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. A method for enriching epileptiform discharges and predicting SOZ during epilepsy interictal period, comprising following steps:
obtaining stereotactic electroencephalogaphy signals of a patient during the epilepsy interictal period;
preprocessing the stereotactic electroencephalogaphy signals to obtain processed stereotactic electroencephalogaphy signals;
dividing the processed stereotactic electroencephalogaphy signals into a training set and a test set, dividing the training set into a plurality of signal segments by using a sliding window, and performing a self-supervised reconstruction training on the signal segments based on a Transformer encoder model to obtain a trained Transformer encoder model; before adopting the sliding window, masking and position coding are to be performed on the sliding window, specifically comprising: masking a middle position of the sliding window with 0; and using sine and cosine functions for position coding; dividing the training set into a plurality of signal segments by adopting the sliding window, and performing the self-supervised reconstruction training on the signal segments based on the Transformer encoder model, and obtaining the trained Transformer encoder model by following method:
$$\begin{matrix}{PE}_{( pos , 2 i )} = sin [ pos / {10000}^{2 i / {d}_{model}} ] \\ {PE}_{( pos , 2 i + 1 )} = cos [ pos / {10000}^{2 i / {d}_{model}} ] \\ Attention ( Q , K , V ) = softmax ( \frac{{QK}^{T}}{\sqrt{{d}_{k}}} ) V\end{matrix}$$
wherein PE represents position coding, pos represents position, i represents dimension, d_{model} represents dimension size, sine function is used for even dimensions, and cosine function is used for odd dimensions; Q is a query vector, K is a vector of correlation between queried information and other information, V is a vector of the queried information, and dₖ is dimension size;
inputting the test set into the trained Transformer encoder model, obtaining reconstructed values of each of the signal segments, comparing the reconstructed values of each of the signal segments with values of the stereotactic electroencephalogaphy signals to obtain deviation values of each of the signal segments from the stereotactic electroencephalogaphy signals; a method for comparing the reconstructed values of each of the signal segments with the values of the stereotactic electroencephalogaphy signals to obtain the deviation values of each of the signal segments from the stereotactic electroencephalogaphy signals is as follows:
$$MSE = \frac{1}{n} {\sum }_{i = 1}^{n} {( {Y}_{i} - {\hat{Y}}_{i} )}^{2} ,$$
wherein MSE is a mean square error function, n is a number of signal points in the signal segments, and Yᵢ is an i-th real signal and \vec{Y}ᵢ is an i-th predicted signal;
setting threshold values of the deviation values, extracting all signal segments exceeding the threshold values of the deviation values, and performing processing based on the signal segments to obtain averaged signal segments; and
inputting the averaged signal segments into a bidirectional long short term memory recursive neural network model to classify and evaluate the stereotactic electroencephalogaphy signals, and completing epileptiform discharge enrichment and SOZ prediction during the epilepsy interictal period.

2. The method for enriching the epileptiform discharges and predicting the SOZ during the epilepsy interictal period according to claim 1, wherein a method for obtaining the stereotactic electroencephalogaphy signals during the epilepsy interictal period of the patient comprises:
placing stereotactic electroencephalogaphy electrodes in the patient by adopting stereotactic technology, setting a sampling rate, and obtaining the stereotactic electroencephalogaphy signals during the epilepsy interictal period of the patient.

3. The method for enriching the epileptiform discharges and predicting the SOZ during the epilepsy interictal period according to claim 1, wherein a method of preprocessing the stereotactic electroencephalogaphy signals to obtain the processed stereotactic electroencephalogaphy signals comprises:
based on the stereotactic electroencephalogaphy signals, adopting a bipolar reference to minimize correlation between two adjacent channels, then performing high-pass filtering on the stereotactic electroencephalogaphy signals, and performing an unified resampling, and finally subjecting the stereotactic electroencephalogaphy signals to Z-score standardization to obtain the processed stereotactic electroencephalogaphy signals.

4. The method for enriching the epileptiform discharges and predicting the SOZ during the epilepsy interictal period according to claim 1, wherein a method for performing the processing based on the signal segments to obtain the averaged signal segments comprises: converting the signal segments by using a smooth nonlinear energy algorithm to obtain converted signal segments; performing average processing on the converted signal segments to obtain the averaged signal segments.

5. The method for enriching the epileptiform discharges and predicting the SOZ during the epilepsy interictal period according to claim 1, wherein inputting the averaged signal segments into the bidirectional long short term memory recursive neural network model to classify the stereotactic electroencephalogaphy signals, wherein the bidirectional long short term memory recursive neural network model introduces a bidirectional propagation mechanism and an attention mechanism on a basis of a long short term memory network, specifically comprising:
$$\begin{matrix}{f}_{t} = σ ( {W}_{f} \cdot [ {x}_{t} , {h}_{t - 1} ] + {b}_{f} ) \\ {i}_{t} = σ ( {W}_{i} \cdot [ {x}_{t} , {h}_{t - 1} ] + {b}_{i} ) \\ {g}_{t} = tanh ( {W}_{c} \cdot [ {x}_{t} , {h}_{t - 1} ] + {b}_{c} ) \\ {c}_{t} = {i}_{t} {g}_{t} + {f}_{t} {c}_{t - 1} \\ {o}_{t} = σ ( {W}_{o} \cdot [ {x}_{t} , {h}_{t - 1} ] + {b}_{o} ) \\ {h}_{t} = {o}_{t} tanh ( {c}_{t} ) \\ {h}_{i} = [ {\vec{h}}_{i} ⊕ {\overleftarrow{h}}_{i} ] ,\end{matrix}$$
wherein σ is a sigmod function, xₜ is an input at a t-th time, and hₜ₋₁ is a hidden layer vector at a last time; fₜ is a forgetting gate, W_{f} is a learning weight of the forgetting gate, and bris a learning weight bias of the forgetting gate; iₜ and gₜ are two branch lines of an input gate, cₜ is an output of the input gate, Wᵢ and W_{c} are learning weights of the input gate, and bᵢ and b_{c} are learning weight biases of the input gate; oₜ is an output gate, Wₒ is the learning weight of the output gate, and bₒ is a learning weight bias of the output gate; hₜ is a hidden layer vector calculated at the current t time; \vec{h}ᵢ and [symbol] are hidden layer vectors from front to back and from back to front, respectively.

6. The method for enriching the epileptiform discharges and predicting the SOZ during the epilepsy interictal period according to claim 1, wherein a method for evaluating the stereotactic electroencephalogaphy signals comprises:
$$\begin{matrix}Accuary = \frac{TP + TN}{TP + FP + FN + TN} \\ Sensitivity = \frac{TP}{TP + FN} \\ Specificity = \frac{TN}{TN + FP}\end{matrix}$$
wherein TP is true positive, TN is true negative, FP is false positive, FN is false negative, Accuracy is accuracy, Sensitivity is sensitivity and Specificity is specificity.
